(19) United States
(12) Patent Application Publication
(10) Pub. No.: US 2007/0053962 A1
(43) Pub. Date: Mar. 8, 2007

(54) Diagnostic markers of wound infection III

(76) Inventors: Rachael L. Clark, Skipton (GB); Breda Mary Cullen, Skipton (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/516,212

(22) Filed: Sep. 6, 2006

(30) Foreign Application Priority Data
Sep. 7, 2005 (GB) 0518288.6

(51) Int. Cl. A61L 15/00; G01N 33/567

(52) U.S. Cl. 424/445; 435/7.2

(57) ABSTRACT

The present invention relates to a method of diagnosis or prognosis of a mammalian wound infection, said method comprising the step of measuring the level of at least one cell surface receptor in a sample of wound fluid. The preferred cell surface receptors are Intercellular adhesion molecule-1 (ICAM 1) and Tumor Necrosis Factor Receptor-2 (TNF-RII). The present invention also provides devices (e.g. biosensors) for use in such methods, and methods and products for diagnosing and treating infected wounds.

### CROSS-REFERENCE TO RELATED APPLICATIONS

This application claims priority from GB 0518288.6 filed on Sep. 7, 2005. All documents cited herein are incorporated by reference in their entirety.

### FIELD OF THE INVENTION

The present invention relates to monitoring patients for the onset or development of wound infection, by detecting the presence and/or level of cell surface receptors (in particular ICAM 1 or TNF-RII) in wound fluid. The present invention provides methods of diagnosis, prognosis and treatment; and also provides wound dressings, apparatus and devices (e.g. wound dressings and biosensors) and kits for use in such methods.

### BACKGROUND OF THE INVENTION

In mammals, injury triggers an organised complex cascade of cellular and biochemical events that result in a healed wound. Wound healing is a complex dynamic process that results in the restoration of anatomic continuity and function; an ideally healed wound is one that has returned to normal anatomic structure, function and appearance.

Chronically contaminated wounds all contain a tissue bacterial flora. These bacteria may be indigenous to the patient or might be exogenous to the wound. Closure, or eventual healing of the wound is often based on a physician's ability to control the level of this bacterial flora. Infection of wounds by bacteria delays the healing process, since bacteria compete for nutrients and oxygen with macrophages and fibroblasts, whose activity are essential for the healing of the wound. Infection results when bacteria achieve dominance over the systemic and local factors of host resistance. Infection is therefore a manifestation of a disturbed host/bacteria equilibrium in favour of the invading bacteria. This elicits a systemic septic response, and also inhibits the multiple processes involved in wound healing. Lastly, infection can result in a prolonged inflammatory phase and thus slow healing, or may cause further necrosis of the wound. The granulation phase of the healing process will begin only after the infection has subsided.

In clinical practice, a diagnosis of infection is based on the presence of local pain, heat, swelling, discharge and redness, although many clinical indicators, such as inflammation and discharge, have a low predictive value of infection in wounds. Definitive diagnosis is achieved by microbiological analysis of wound samples. Tissue biopsy provides the most accurate results, but this is an invasive procedure that is difficult to achieve for the mass of specimens required. Wound swabbing is the most common wound sampling method used in the UK although its clinical value has been questioned. Furthermore, microbiological diagnosis of wound infection can take 48 to 72 hours, which allows time for infection to further develop if first-line/best-guess treatment is not employed immediately.

There therefore remains a need in the art for a method for the early diagnosis and prognosis of wound infection, and for devices and wound dressings for use in carrying out such methods.

### SUMMARY OF THE INVENTION

The present inventors have made the surprising discovery that elevated levels of cell surface receptors, in particular ICAM 1 and TNF-RII, are indicative of wound infection, and that the level of the said cell surface receptors in wound fluid is correlated to the level of bioburden in the wound. The present inventors have found that increase in wound fluid total bioburden correlates to an increase in the concentration of certain cell surface receptors, in particular ICAM 1 and TNF-RII in the wound fluid. This data suggests that ICAM 1 and TNF-RII may be important markers of infection in wounds.

### BRIEF DESCRIPTION OF THE DRAWINGS

FIG. 1 shows a log-log graph of the measured TNF-RII level (in pg/ml) against total bioburden (in cfu/ml) for a range of venous leg ulcer wound fluids, with a best-fit linear regression; and

FIG. 2 shows a log-log graph of the measured ICAM level (in pg/ml) against total bioburden (in cfu/ml) for a range of venous leg ulcer wound fluids, with a best-fit linear regression.

### DETAILED DESCRIPTION OF THE INVENTION

Intercellular adhesion molecule-1 (hereinafter ICAM-1) is a cytokine-inducible cell surface receptor that binds to leukocyte integrins LFA-1 and Mac-1.

Tumor Necrosis Factor Receptor 2 (hereinafter TNF-RII) mediates similar actions to the pro-inflammatory cytokine TNF-alpha, such as T cell proliferation and cytokine production as well as other actions, such as the migration of Langerhans cells to lymph nodes. In normal skin, TNF-RII is not found in the epidermis, and is only associated with eccrine sweat ducts and dermal dendritic cells.

The present invention relates to cell surface receptors as target molecules in a new diagnostic or prognostic assays to monitor patients for wound infection. Standard diagnostic technology (e.g. immunodetection) can be used to detect the cell surface receptors. Antibodies which detect cell surface receptors directly or indirectly may be employed and are available commercially. The inventive assays may be laboratory-based, or they may be carried out at the point of care.

Accordingly, in a first aspect the present invention provides a method of diagnosis or prognosis of infection of a mammalian wound, said method comprising the step of measuring the level of at least one cell surface receptor in a sample of wound fluid from said wound.

The finding that cell surface receptors are elevated in infected fluid means that the differentiation between infected and basal levels is significant. Cell surface receptors and associated breakdown products or fragments thereof in wound fluid are therefore used as a diagnostic or prognostic of wound infection. Accordingly, in a further aspect, the method of the present invention comprises comparing the measured level of the one or more cell surface receptors in the sample of wound fluid with a reference level characteristic of a non-infected wound.

In further embodiments, the method comprises sampling the wound fluid at intervals of from about 1 hour to about 24 hours and measuring the level of the one or more cell surface receptors in the samples obtained at said intervals. This allows the evolution of the level of the one or more cell surface receptors over time to be observed. An increase in the cell surface receptors over time would be strongly indicative of developing infection.

Suitably, the method according to the present invention further comprises measuring a total protein content of the wound fluid and normalising the measured levels of cell surface receptors to constant total protein content. This allows variation in the rate of production and concentration of the wound fluid to be corrected.

Suitably, the cell surface receptors are selected from the group consisting of adhesion molecules, lectins and TNF-RII. Examples of suitable adhesion molecules and lectins include ICAM-1, ICAM-2, VCAM-1, E-selectin, P-selectin and L-selectin and E-cadherin. Preferably, the cell surface receptors are selected from the group consisting of ICAM 1 and TNF-RII.

In a second aspect, the present invention provides a method for the treatment of a mammalian wound comprising the steps of: measuring the level of one or more cell surface receptors in a sample of wound fluid, and applying an antimicrobial wound dressing to the wound selectively if the presence or level of said one or more cell surface receptors is indicative of wound infection.

In certain embodiments according to this aspect, the method further comprises applying a wound dressing that is substantially free of antimicrobial agents to the wound if the level of said one or more cell surface receptors is indicative of absence of wound infection.

In certain embodiments according to this aspect, the method further comprises sampling the wound fluid at intervals, for example at intervals of from 1 hour to 24 hours, and selecting an antimicrobial or non-antimicrobial dressing to treat the wound at said intervals in response to the measured presence or level of said cell surface receptors, or in response to changes in the measured level of the level of said one or more cell surface receptors. For example, the antimicrobial wound dressing may be applied to the wound if the said level is increasing over time, and the non-antimicrobial dressing is applied to the wound if the said level is decreasing over time.

The diagnostic and prognostic methods may be performed on wound fluid that has been removed from the body (e.g. as a clinical swab or as a fluid sample) but can also be performed in situ. The decision as to which method is used will depend upon the type of wound being studied.

The test on the fluid sample may be qualitative. Alternatively, a quantitative or semi-quantitative test for the marker may be performed. Thus, in one embodiment the concentration of the one or more cell surface receptors is measured.

Various methods may be used to detect or measure the concentration of the one or more cell surface receptors. Suitable methods include those utilising chemical or enzyme-linked reactions, or immunological methods (e.g. ELISA, western blots), spectrophotometric, colorimetric, fluorimetric, or radioactive detection based techniques. In one embodiment a dip-stick type test is provided. Such a test could be used in the community and by the patient allowing easier and earlier diagnosis/prognosis.

For example, in the case of surface-exposed wounds, a clinical swab, dressing, “dipstick” or other biosensor device may be applied directly to the surface of the wound. The device can then be removed from the wound and the one or more cell surface receptors measured by the appropriate means. In many cases, a physician may not actually require an accurate assessment of the precise concentration of the marker, but may just wish to know whether there is a sufficient concentration of the marker to warrant prophylactic or curative action as necessary. In these cases, visible assessment of the dressing may be sufficient to allow identification of the specific areas of infection. Unnecessary treatment of healthy granulating tissue can then be avoided.

A dressing that allows mapping of the infected areas of a wound will be preferable in certain instances. Diagnostic wound mapping sheets that could be adapted to the methods of the present invention are described in GB-A-2323166 (application no. GB 9705081.9), filed on 12 Mar. 1997, the entire content of which is hereby incorporated by reference.

The devices, of the present invention comprise selective binding partner, preferably a specific binding partner, for example respective immunological binding partners for the one or more cell surface receptors. Suitable immunological binding partners include antibodies, including both polyclonal antibodies and monoclonal antibodies. Examples of suitable antibodies which may be employed in the present invention include: anti-ICAM-1 from R&D Systems (Catalogue No. BBE1B Range 2.73-49.55 ng/ml Sensitivity <0.35 ng/ml); anti-E-Selectin from R&D Systems (Catalogue No. BBE2B Range 0.47-10.52 ng/ml Sensitivity <0.1 ng/ml); anti-L-Selectin from R&D Systems (Catalogue No. BBE4B Range 0.99-57.72 ng/ml Sensitivity <0.3 ng/ml); anti-P-Selectin from R&D Systems (Catalogue No. BBE6 Range 0.82-45.94 ng/ml Sensitivity <0.5 ng/ml); anti-E-cadherin from (R&D systems Catalogue No. DCADEO Range 0.31-20 ng/ml Sensitivity 0.04 ng/ml); anti-TNF receptor II (anti-TNF-RII) from R&D systems (Catalogue No. DTR200 Range 7.8-500 pg/ml Sensitivity 0.6 pg/ml); and anti-VCAM-1 from R&D systems (Catalogue No. DVC00 Range 6.25-200 ng/ml Sensitivity 0.6 ng/ml).

Other suitable antibodies which may be employed as immunological binding partners in the present invention are any antibodies which are immunospecific for the monitored marker. In accordance with the invention the monitored marker is one or more cell surface receptors, or fragments or breakdown products thereof. Such fragments include low molecular weight peptides that are derived from the one or more cell surface receptor peptides as a consequence of protease activity. Breakdown products of the one or more cell surface receptors may be recognised by polyclonal antibodies that have been raised to the one or more cell surface receptors.

Alternatively or additionally, the marker is a host-derived moiety which interacts with the one or more cell surface receptors. The moiety may, for example, bind to the one or more cell surface receptors. Preferably, the binding is specific. In one embodiment of the invention the moiety interacts with the one or more cell surface receptors.

By a moiety which interacts with the one or more cell surface receptors we also include breakdown products of said moeities, e.g. fragments of the said moeity.

The term “immunospecific” means that the antibodies have substantially greater affinity for the monitored marker than their affinity for other molecules related to the monitored markers. By “substantially greater affinity” we mean that there is a measurable increase in the affinity for the molecular marker as compared with the affinity for other molecules related to the monitored marker. Preferably, the affinity is at least 1.5-fold, 2-fold, 5-fold 10-fold, 100-fold, 10³-fold, 10⁴-fold, 10⁵-fold, 10⁶-fold or greater for the monitored marker than for other molecules related to the monitored marker.

If polyclonal antibodies are desired, a selected mammal, such as a mouse, rabbit, goat or horse, may be immunised with the monitored marker. The monitored marker used to immunise the animal can be obtained by any suitable technique, for example, it can be purified from a wound fluid sample from an infected wound, it can be derived by recombinant DNA technology or it can be synthesized chemically. If desired, the monitored marker can be conjugated to a carrier protein. Commonly used carriers to which the monitored markers may be chemically coupled include bovine serum albumin, thyroglobulin and keyhole limpet haemocyanin. The optionally coupled monitored marker is then used to immunise the animal. Serum from the immunised animal is collected and treated according to known procedures, for example by immunoaffinity chromatography.

Monoclonal antibodies to the monitored marker can also be readily produced by one skilled in the art. The general methodology for making monoclonal antibodies using hybridoma technology is well known.

Panels of monoclonal antibodies produced against the monitored marker can be screened for various properties, i.e., for isotype, epitope, affinity, etc. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions. Humanised antibodies may also be used. The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is, an antibody having two different antigen binding domains, each domain being directed against a different epitope.

Phage display technology may be utilised to select genes which encode antibodies with binding activities towards the monitored marker either from repertoires of PCR amplified V-genes of lymphocytes from humans screened for possessing the relevant antibodies, or from naive libraries. The affinity of these antibodies can also be improved by chain shuffling.

Where antibodies generated by the above techniques, whether polyclonal or monoclonal, are employed as reagents in immunoassays, radioimmunoassays (RIA) or enzyme-linked immunosorbent assays (ELISA), the antibodies can be labelled with an analytically-detectable reagent such as a radioisotope, a fluorescent molecule or an enzyme.

As used herein, the term “antibody” refers to intact molecules as well as to fragments thereof, such as Fab, F(ab′)2 and Fv, which are capable of binding to the antigenic determinant in question. Such antibodies thus bind to the monitored marker.

The immunological binding partner may be immobilized or bound to a solid substrate in a device as described herein. Immobilisation of reaction components, in particular of selective binding partners for the one or more cell surface receptors, onto a dipstick, wound mapping sheet or other solid or gel substrate offers the opportunity of performing a more quantitative measurement. For example, in the case of a reaction linked to the generation of a colour the device may be transferred to a spectrometer. Suitable methods of analysis will be apparent to those of skill in the art.

Immobilisation of the reaction components to a small biosensor device will also have the advantage that less of the components (such as antibody, enzyme and substrate) are needed. The device will thus be less expensive to manufacture than a dressing that needs to have a large surface area in order to allow the mapping of a large wound area.

Methods for the incorporation of the components of the assay reaction onto a clinical dressing, “dipstick”, sheet or other biosensor are routine in the art. See for example Fägerstam and Karlsson (1994) Immunochemistry, 949-970.

The detectable signal produced by the device according to the present invention is observable or measurable by a physical, chemical, or biological means known to those of skill in the art. A detectable signal may be a change in emission or absorbance of electromagnetic waves at a certain wavelength, hybridization or enzymatic reaction. In preferred embodiments, detectable signals are changes in colour when viewed under white light, or fluorescence when viewed under UV light. In certain embodiments, the device may comprise an electronic sensor, for example to detect color change or fluorescence and to provide a quantitative output thereof. The device may include an electronic sensor that can provide a quantitative output in digital form.

The device may further comprise a reference assay element for determining the total protein content of the sample, so that the measured level of marker can be normalised to constant total protein level in order to increase accuracy.

In certain embodiments, the device according to the present invention comprises, or consists essentially of a wound dressing, dipstick or swab. In certain embodiments, the device according to the present invention comprises a housing containing one or more reagents and having an inlet provided therein for introduction of the sample. The housing may be at least partially transparent, or may have windows provided therein, for observation of an indicator region that undergoes a color or fluorescence change. In certain embodiments, the device operates on the lateral flow principle. That is to say, said device comprises a housing having an inlet for the sample and side walls defining a fluid lateral flow path extending from the inlet. By “lateral flow”, it is meant liquid flow in which the dissolved or dispersed components of the sample are carried, preferably at substantially equal rates, and with relatively unimpaired flow, laterally through the carrier. Suitably, the fluid flow path contains one or more porous carrier materials. The porous carrier materials are preferably in fluid communication along substantially the whole fluid flow path so as to assist transfer of fluid along the path by capillary action. Suitably, the porous carrier materials are hydrophilic, but preferably they do not themselves absorb water. The porous carrier materials may function as solid substrates for attachment of reagents or indicator moieties. In certain embodiments of the present invention, the device further comprises a control moiety located in a control zone in said in said device, wherein the control moiety can interact with a component of the wound fluid sample to improve the accuracy of the device.

The size and shape of the carrier are not critical and may vary. The carrier defines a lateral flow path. Suitably, the porous carrier is in the form of one or more elongate strips or columns. In certain embodiments, the porous carrier is one or more elongate strips of sheet material, or a plurality of sheets making up in combination an elongate strip. One or more reaction zones and detection zones would then normally be spaced apart along the long axis of the strip. However, in some embodiments the porous carrier could, for example be in other sheet forms, such as a disk. In these cases the reaction zones and detection zones would normally be arranged concentrically around the center of the sheet, with a sample application zone in the center of the sheet. In yet other embodiments, the carrier is formed of carrier beads, for example beads made from any of the materials described above. The beads may suitably be sized from about 1 micrometer to about 1 mm. The beads may be packed into the flow path inside the housing, or may be captured or supported on a suitable porous substrate such as a glass fiber pad.

It will be appreciated that the devices according to the present invention may be adapted to detect more than one marker or other analyte. This can be done by the use of several different reagents in a single reaction zone, or preferably by the provision in a single device of a plurality of lateral flow paths each adapted for detecting a different analyte. In certain embodiments, the plurality of lateral flow paths are defined as separate fluid flow paths in the housing, for example the plurality of lateral flow paths may be radially distributed around a sample receiving port. In some embodiments, the plurality of fluid flow paths are physically separated by the housing. In other embodiments multiple lateral flow paths (lanes) can be defined in a single lateral flow membrane by depositing lines of wax or similar hydrophobic material between the lanes.

The devices according to the present invention may for example be incorporated into a bacterial sensing device of the kind described in copending application GB 0501818.9 filed on 28 Jan. 2005, the entire content of which is incorporated herein by reference.

An absorbent element may suitably be included in the devices of the present invention. The absorbent element is a means for drawing the whole sample through the device by capillary attraction. Generally, the absorbent element will consist of a hydrophilic absorbent material such as a woven or nonwoven textile material, a filter paper or a glass fiber filter.

The device may further comprise at least one filtration element to remove impurities from the sample before the sample undergoes analysis. The filtration device may for example comprise a microporous filtration sheet for removal of cells and other particulate debris from the sample. The filtration device is typically provided upstream of the sample application zone of the fluid flow path, for example in the inlet of the housing or in the housing upstream of the inlet.

Preferably, the devices according to the present invention include a control moiety in a control zone of the device, wherein the control moiety can interact with a component of the wound fluid sample to improve the accuracy of the device. Suitably, the control zone is adapted to reduce false positive or false negative results. A false negative result could arise for various reasons, including (1) the sample is too dilute, or (2) the sample was too small to start with.

In order to address false negative mechanism, the control zone preferably further comprises a reference assay element for determining the total protease content or the total protein content of the sample, that is to say for establishing that the total protease content or the total protein content of the sample is higher than a predetermined minimum. It is possible to indicate the presence of protein by the use of tetrabromophenol blue, which changes from colorless to blue depending on the concentration of protein present. It is also possible to detect glucose (using glucose oxidase), blood (using diisopropyl-benzene dihydro peroxide and tetramethylbenzidine), leukocytes (using ester and diazonium salt). These may all be useful analytes for detection in the control zone for the reduction of false negatives.

In a further aspect, the present invention provides a diagnostic kit comprising a diagnostic device according to the present invention. The test system or kit may comprise, in addition to a diagnostic device according to the present invention, one or more components selected from: a color chart for interpreting the output of the diagnostic device, a sampling device for collecting a sample of a biological fluid such as a wound fluid, a wash liquid for carrying a sample of fluid through the device, and a pretreatment solution containing a reagent for pretreatment of the fluid sample.

Where present, the sampling device may comprise a swab or a biopsy punch, for example a shaft having a swab or biopsy punch attached thereto. Suitably, the diagnostic device includes a sample receiving port, and preferably the sample receiving port and the swab or biopsy punch comprise complementary fitting elements whereby the swab or biopsy punch can be secured to the device with the swab or biopsy punch received in the sample receiving port.

In certain embodiments the fitting element on the shaft may be located from 1 mm to about 30 mm from the base of the swab or the biopsy punch. This is consistent with the use of relatively small sample receiving port on the housing of the diagnostic device. The sample receiving port is typically located on an upper surface of the diagnostic device, and it is typically generally in the form of an upwardly projecting tube, open at the top and having the inlet to the fluid flow path located at the bottom of the tube. Suitable swabs, biopsy punches and sample receiving caps are described in detail in copending applications GB0403976.4 and GB0403978.0 both filed on 23 Feb. 2004, the entire contents of which are incorporated herein by reference.

The fitting element on the shaft may a tapered region of the shaft for forming an interference fit with the housing, for example it may appear as a truncated cone that is coaxial with the shaft and tapers towards the first end of the shaft. Or the whole shaft may have a diameter larger than that of the swab or biopsy punch, with a tapered region adjacent to the first end. In any case, the diameter of the tapered region where it engages with the housing is normally greater than the diameter of the swab or biopsy punch, so that the inlet port can enclose the swab or biopsy punch.

In other embodiments, the engagement element may comprise a snap-fitting projection for forming a snap-fit with one or more complementary projections on an inner surface of the housing, or a threaded projection for forming a screw fit with one or more complementary threads on an inner surface of the cap, or a Luer-lock type fitting.

The swab may be any absorbent swab, for example a nonwoven fibrous swab. Typically the diameter of the swab is about 2 to about 5 mm, for example about 3 mm. In certain embodiments, the swab may be formed from a medically acceptable open-celled foam, for example a polyurethane foam, since such foams have high absorbency and can readily be squeezed to expel absorbed fluids. The biopsy punch will typically be a stainless steel cylindrical punch of diameter about 1 mm to about 10 mm, for example about 3 mm to about 8 mm, suitably about 6 mm.

In certain embodiments the shaft is hollow, whereby a fluid can be passed down the shaft from the second end to expel the biological sample from the swab or the biopsy punch into the diagnostic device. This helps to ensure that all of the sample passes through the device, thereby avoiding false negatives. The shaft may comprise a fitting at the second end for attachment of a syringe or other source of the fluid. In certain embodiments, the apparatus may comprise a reservoir of liquid attached to the second end of the shaft, for example a compressible bulb containing the liquid, which can be activated after use of the swab or biopsy punch. Suitable devices of this kind are described, for example in U.S. Pat. No. 5,266,266, the entire content of which is incorporated herein by reference. In other embodiments, the apparatus may comprise a plunger that can be pushed down the hollow bore of the shaft to expel fluid or other specimens from the swab or biopsy punch.

Another advantage of the hollow shaft is that, where the apparatus is a biopsy punch, the biopsy sample can more readily be pushed or blown out of the punch. The biopsy punch apparatus can further comprise a homogenizing tool that can be passed down the hollow shaft to homogenize a tissue sample in the biopsy punch. This step of homogenizing can be followed, if necessary, by passing liquid down the shaft from the second end to expel the homogenized tissue from the biopsy punch into the device for diagnostic analysis.

In this aspect of the invention, the swab or biopsy punch may be sterilized, and may be packaged in a microorganism-impermeable container. The diagnostic devices according to the present invention may also be sterilized, but they may not, because the devices often do not come into contact with the patient being diagnosed.

The concentration of the marker may be measured in an aqueous assay system. For instance, wound fluid may be extracted directly from the environment of the wound or can be washed off the wound using a saline buffer. The resulting solution can then be assayed for the concentration of the marker in, for example, a test tube or in a microassay plate.

Such a method will be preferable for use in cases in which the wound is too small or too inaccessible to allow access of a diagnostic/prognostic device such as a dipstick. This method has the additional advantage that the wound exudate sample may be diluted.

It will be clear that an aqueous assay system is more applicable to use in a laboratory environment, whereas a wound dressing containing the necessary reaction components will be more suitable for use in a hospital or domestic environment.

The diagnosis, prognosis or treatment according to the present invention may be performed on any type of wound. For example, the wound may be an acute wound such as an acute traumatic laceration, perhaps resulting from an intentional operative incision, or the wound may be a chronic wound. Examples of chronic wounds include venous ulcers, pressure sores, decubitis ulcers, diabetic ulcers and chronic ulcers of unknown aetiology.

In a further aspect, the invention also provides a system for use in the diagnosis and treatment of wounds comprising a diagnostic device according to the invention and a wound dressing comprising at least one antimicrobial agent. The wound dressing comprising the antimicrobial agent(s) can be applied to the wound selectively, when the diagnostic device indicates the presence of wound infection.

Preferably, the system according to this aspect further comprises a wound dressing that is substantially free from antimicrobial agents, for application to the wound when the measured presence or level of a marker is indicative of a non-infected wound. The system may be in the form of a kit, and the device and the wound dressing(s) may be packaged together in a single package.

These aspects of the invention avoid unnecessary application of antimicrobial agents to the wound, which is desirable because most antimicrobial agents are cytotoxic and interfere with wound healing, and also to avoid the development of resistant microorganisms.

The antimicrobial wound dressing used in these aspects of the invention comprises an effective amount of an antimicrobial agent, which may preferably be selected from the group consisting of antiseptics and antibiotics and mixtures thereof. Suitable antibiotics include peptide antimicrobials (e.g. defensins, Magainin, synthetic derivatives of them) tetracycline, penicillins, terramycins, erythromycin, bacitracin, neomycin, polymycin B, mupirocin, clindamycin and mixtures thereof. Suitable antiseptics include silver sulfadiazine, chlorhexidine, povidone iodine, triclosan, other silver salts and colloidal silver, sucralfate, quaternary ammonium salts and mixtures thereof.

The wound dressing materials used in these aspects of the invention may for example be provided in the form of beads, flakes, powder, and preferably in the form of a film, a fibrous pad, a web, a woven or non-woven fabric, a freeze-dried sponge, a foam or combinations thereof. In certain embodiments, the dressing material is selected from the group consisting of woven fabrics, knitted fabrics, and nonwoven fabrics, all of which may be made by conventional methods. In other embodiments, the material may comprise (or consist essentially of) a freeze-dried sponge or a solvent-dried sponge.

The wound dressing material may be in the form of a solid, or a semi-solid ointment or gel. Preferably, the wound dressing material comprises only up to 20% by weight, preferably less than 10% by weight of water. The relatively low water content improves the stability of the material and makes it possible to sterilize by heat or irradiation without loss of activity. The material may also contain 0-40% by weight, preferably 0-25% by weight of a plasticiser, preferably a polyhydric alcohol such as glycerol. All of the above percentages are on a dry weight basis.

The present invention may be used in the diagnosis, prognosis and treatment of inflammatory conditions and infections in human and non-human mammals.

As used herein, the term wound fluid is meant to refer to the exudate that is secreted or discharged by cells in the environment of the wound. The term “wound fluid” herein refers to any wound exudate or other fluid (preferably substantially not including blood) that is present at the surface of the wound, or that is removed from the wound surface by aspiration, absorption or washing. The term “wound fluid” does not normally refer to blood or tissue plasma remote from the wound site.

By an elevated or reduced level of the one or more cell surface receptors we include where the level of the marker is significantly higher or lower respectively than basal/normal levels of the marker and is thereby indicative of an infection or other inflammatory condition.

The level of the one or more cell surface receptor markers is significantly higher or lower than the control (normal level) if the level of the marker is greater or lower than the control level by an amount greater than the standard error of the assay employed to assess expression, and preferably at least twice, and more preferably three, four, five or ten times that amount. Alternately, the level of the marker can be considered “significantly” higher or lower than the control level of the marker if the marker is at least about 1.5, two, three, four, or five times, higher or lower, respectively, than the control level of the marker.

### EXAMPLE 1

Collection and Treatment of Wound Fluid—Removal of Infected and Non-Infected Wound Fluid

All patients enrolled in the study had venous leg ulcers of at least 30 days duration and a surface area of at least 1 cm². Patients were diagnosed as ‘non-infected, normal appearance of wound, or ‘infected’ based on a minimum of 4 clinical signs and symptoms indicative of infection. Patients were excluded from the study if exposed bone with positive osteomyelitis was observed. Other exclusion criteria included concomitant conditions or treatments that may have interfered with wound healing and a history of non-compliance that would make it unlikely that a patient would complete the study. Wound fluids were collected from the patients following informed consent being given from all patients or their authorized representatives. The protocol was approved by the Ethics Committee at the participating study center prior to commencement of the study. The study was conducted in accordance with both the Declaration of Helsinki and Good Clinical Practice.

Cell Surface Receptor Assays

The wound fluid samples were analysed using ELISA assays employing either the anti-ICAM-1 antibody from R&D Systems (Catalogue No. BBE1B Range 2.73-49.55 ng/ml Sensitivity <0.35 ng/ml) or the anti-TNF-RII antibody from R&D systems (Catalogue No. DTR200 Range 7.8-500 pg/ml Sensitivity 0.6 pg/ml). ELISA assays were performed using kits purchased from R&D Systems in accordance with the instructions provided therewith.

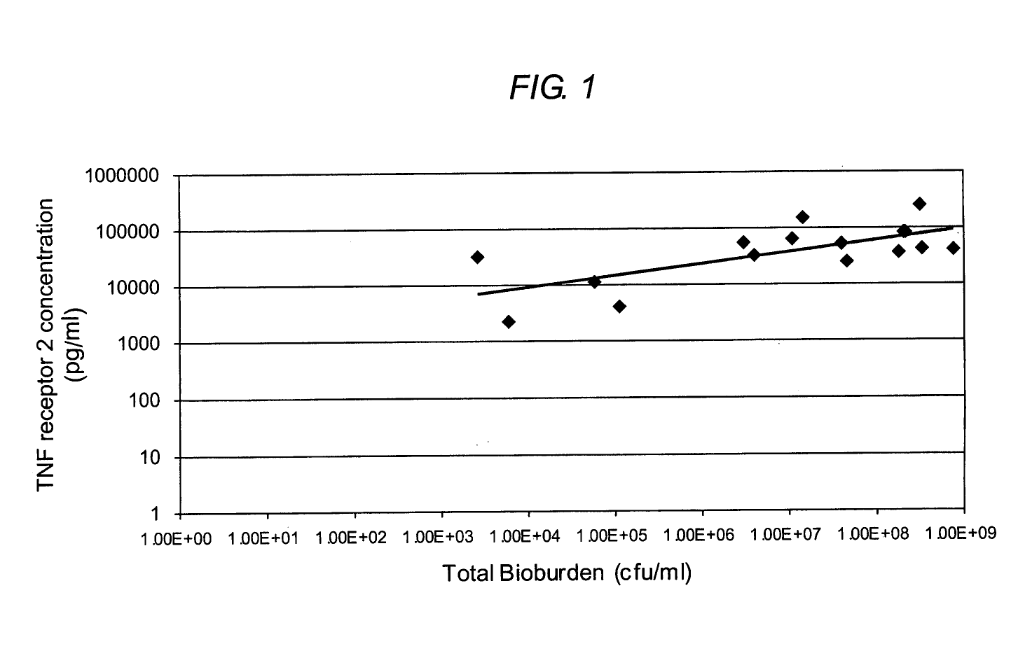

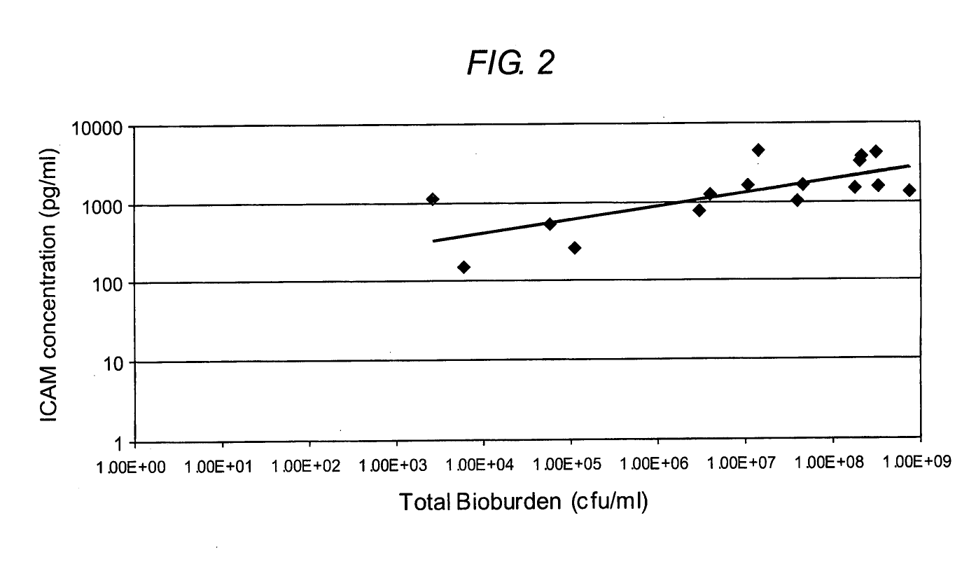

The results of the assays are shown in FIGS. 1 and 2. It can be seen that the levels of both ICAM 1 and TNF-RII increase with bacterial bioburden by approximately an order of magnitude over the range of bioburden studied.

The above examples have been described by way of example only. Many other examples falling within the scope of the accompanying claims will be apparent to the skilled reader.

1. A method of diagnosis or prognosis of a mammalian wound infection, said method comprising the step of measuring the level of at least one cell surface receptor in a sample of wound fluid.

2. A method according to claim 1 wherein the cell surface receptor is selected from the group consisting of ICAM 1, TNF-RII, and combinations thereof.

3. A method according to claim 2 wherein the method is an in vitro method carried out on a sample of wound fluid that has been removed from a patient.

4. A method according to claim 2, wherein said step of measuring comprises contacting the sample of wound fluid with an immunological binding partner for a cell surface receptor.

5. A method according to claim 4, further comprising comparing the measured level of said one or more cell surface receptors in said sample of wound fluid with a reference level characteristic of a non-infected wound.

6. A method according to claim 5, wherein the method comprises sampling the wound fluid at intervals of from about 1 hour to about 24 hours and measuring the level of said one or more cell surface receptors in the samples obtained at said intervals.

7. A method according to claim 5, wherein the method further comprises measuring a total protein content of the wound fluid and normalising the measured levels of said one or more cell surface receptors to constant total protein content.

8. A method for the treatment of a mammalian wound comprising the steps of measuring the level of one or more cell surface receptors selected from the group consisting of ICAM 1, TNF-RII in a sample of wound fluid, and applying an antimicrobial wound dressing to the wound selectively if the presence or level of said marker is indicative of wound infection wherein the step of measuring the level of one or more cell surface receptors comprises contacting the sample of wound fluid with an immunological binding partner for a cell surface receptor comparing the measured level of said one or more cell surface receptors in said sample of wound fluid with a reference level characteristic of a non-infected wound.

9. A method according to claim 8, further comprising applying a wound dressing that is substantially free of antimicrobial agents to the wound if the said presence or level is indicative of absence of wound infection.

10. A method according to claim 8, wherein the method comprises sampling the wound fluid at intervals, for example at intervals of from 1 hour to 24 hours, and selecting an antimicrobial or non-antimicrobial dressing to treat the wound at said intervals in response to the measured presence or level of said marker.

11. A method according to claim 10, wherein the antimicrobial wound dressing is applied to the wound if said level is increasing over time, and the non-antimicrobial dressing is applied to the wound if said level is decreasing over time.
